(19) United States
(12) Patent Application Publication
(10) Pub. No.: US 2020/0330743 A1
(43) Pub. Date: Oct. 22, 2020

(54) APPARATUS FOR TREATING SLEEP APNEA AND SNORING

(71) Applicant: Klaus DUERING, Frechen (DE)

(72) Inventors: Klaus DUERING, Frechen (DE); Joachim Georg PFEFFER, Aachen (DE)

(21) Appl. No.: 16/920,697

(22) Filed: Jul. 4, 2020

Related U.S. Application Data
(63) Continuation of application No. 13/386,960, filed on Feb. 15, 2012.

(30) Foreign Application Priority Data
Jul. 31, 2009 (DE) 202009010388.9

(51) Int. Cl. A61M 39/28; A61F 5/56

(52) U.S. Cl. CPC A61M 39/284; A61F 2/848; A61F 5/56

(57) ABSTRACT

A kit for treating sleep apnea and snoring is described, comprising an insertion tube effective for inserting an apparatus for treating sleep apnea and snoring through a nasal passage into a pharyngeal zone, and an apparatus for treating sleep apnea and snoring, configured for use with the insertion tube so that the apparatus is insertable by the insertion tube through the nasal passage into the pharyngeal zone.

### CROSS-REFERENCE TO RELATED APPLICATIONS

This is a continuation under 35 USC § 119 of U.S. patent application Ser. No. 13/386,960 filed Jan. 25, 2012 as a U.S. national phase under the provisions of 35 U.S.C. § 371 of International Patent Application No. PCT/EP2010/004687 filed Jul. 30, 2010, which in turn claims the benefit of priority under 35 USC § 119 of German Patent Application No. 202009010388.9 filed Jul. 31, 2009. The disclosures of U.S. patent application Ser. No. 13/386,960, International Patent Application No. PCT/EP2010/004687 and German Patent Application No. 202009010388.9 are hereby incorporated herein by reference in their respective entireties, for all purposes.

The present invention relates to a fixing apparatus for fixing an apnea stent in the respiratory duct.

Disease patterns which are based on the at least partial occlusion of cavities, organ ducts and/or vessels, have been constantly increasing because of today's lifestyle. An important sector of such disease patterns relates to the respiratory ducts. For example, obstructive sleep apnea is a life-threatening disease which results from an occlusion of the posterior respiratory ducts due to muscular fatigue during sleep. Repeated, partially very frequent short-term apneas lead to an insufficient supply of the patient with oxygen during sleep. In an extreme case, this may be fatal. Factors caused by today's lifestyle, such as severe obesity or excessive alcohol consumption, markedly increase the disease risk. Snoring shows less severe effects compared to the sleep apnea and results from the same organic causes. While snoring is known to most people, the severe health endangering consequences of heavy snoring and sleep apnea are often not realized since the patient is not aware of the consequences resulting from the organic deficiency symptoms.

WO 2007/065408 A2 discloses an apparatus for splinting and/or keeping open a cavity, an organ duct and/or a vessel in the human or animal body by means of at least one compressible and self-expanding stent which has at least one enlarged area. This stent is fixed using an attachment plate which is not described in more detail against an excessively deep penetration of the respiratory duct. Reference is herewith made to the full content of WO 2007/065408 A2. A stent as described in WO 2007/065408 A2 is referred to below as apnea stent.

This apnea stent has shown very good results in first clinical tests for treating obstructive sleep apnea.

US 2006/00 70 626 A1 relates to a method and an apparatus for treating sleep apnea. The apparatus comprises an endotracheal stent which can be introduced into the trachea by means of endoscopy. The proximal and distal ends of this stent can be provided with hooks, thorns or clips, for example, to arrange the stent in the trachea.

DE 10 2004 012 351 A1 discloses an apparatus for the revascularization of a cavity, organ duct or vessel. The apparatus for revascularization is an expandable stent.

DE 102 40 725 A1 describes an apparatus for preventing snoring and apneas during sleep. This apparatus also describes a type of stent whose end, which protrudes from the nose and which serves the purpose of locking, has an arch shape and is provided with an inlet opening so as not to impair the air supply through the nose.

US 2009/00 44 814 A1 describes a barbed hook system which is provided with hooks that can be locked in the tissue and is implanted by means of surgery.

DE 202 16 956 UI discloses an apparatus for improving nasal breathing and preventing snoring. This apparatus comprises two short tubular members which are linked to each other via a joining member and can be introduced into the alar wings of the nose so as to prevent snoring and improving nasal breathing. This apparatus is only used at the nostril and does not extend into the trachea.

WO 98/23233 A1 describes a tube serving for preventing sleep apnea.

WO 2008/0 58 367 A1 discloses an extendable apparatus which can be inserted in the nose. This apparatus comprises two spiral wound tubular members which can be inserted in the alar wings of the nose and which are linked to each other by means of a joining member.

US 61 06 548 A describes a stent which can be compressed and expanded only once.

US 2001/00 44 647 A1 discloses an expandable endoluminal stent.

DE 102 33 862 A1 describes a system for connecting a tubular line, in particular a plastic hose, with a connecting apparatus. The connecting apparatus has clips which extend in the longitudinal direction and which engage corresponding lugs of the tubular line.

EP 05 17 000 A2 discloses a hose clamp for medical purposes to pinch off a tube.

DE 10 2004 023 559 A1 describes a cardiac catheter including a corresponding stent.

EP 1 181 906 B1 discloses a catheter for introducing a stent.

DE 15 (DE 100 12852 A1) also describes a catheter for introducing expandable stents.

It is the object of the present invention to create an inexpensive apparatus which prevents a penetration of an apnea stent into the respiratory duct after its insertion and/or during sleep in a reliable and safe way and which can be easily handled.

According to the invention, a fixing apparatus is provided to fix an apnea stent in the respiratory duct. This fixing apparatus has a fixing device which can be fixed to a proximal end of the stent so as to fix the position of the stent in the respiratory duct. The fixing apparatus is distinguished by the fixing device having two clamping members between which the proximal end of the apnea stent can be fixed.

This fixing device can be easily handled by means of one hand and therefore the proximal end of the apnea stent can be held with the other hand while fixing the fixing device to the apnea stent. Ease of handling is very important since the position of the proximal end of the apnea stent is located on the edge or outside the field of vision of the respective person using the apnea stent.

An excessively deep penetration into the respiratory duct of a patient is safely and reliably prevented since the fixing apparatus abuts against the alar wings of the patient's nose and the stent thus cannot slip deeper into the respiratory duct of a patient.

Likewise the stent can be safely and reliably prevented from slipping out of the respiratory duct by an attachment of the fixing apparatus to the head.

In a preferred embodiment of the present invention, the fixing apparatus has first and second clamping jaws which are connected to each other via a joint. The two clamping jaws can be arranged so as to abut such that a proximal end of the apnea stent can be fixed between the two jaws. The two clamping jaws can preferably be fixed to each other by means of a locking or snap-action device.

A simple fixation of the apnea stent to the respiratory duct is ensured by the two clamping jaws. The clamping jaws also enable a rapid and easy attachment and release of the fixing apparatus to the proximal end of the stent.

On account of the simple design, the fixing apparatus can be produced easily and inexpensively.

The invention is specified by way of example using the drawings which show by way of diagram:

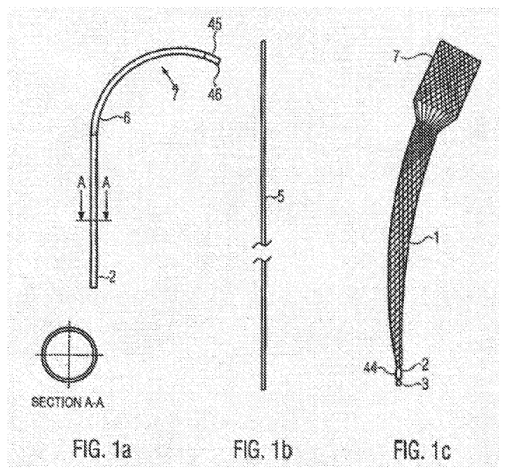

FIG. 1a, b, c a perspective view of an apnea stent together with an insertion bar and an insertion tube,

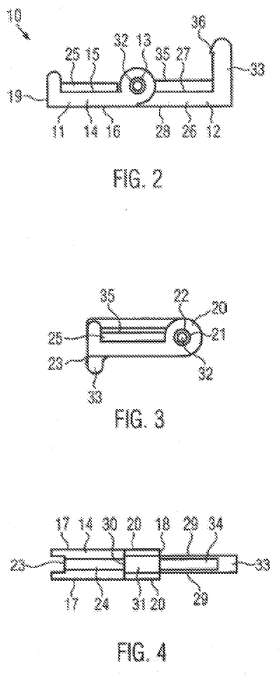

FIG. 2 a side view of a fixing apparatus according to the invention in an open state,

FIG. 3 a side view of a fixing apparatus of FIG. 2 in a closed state,

FIG. 4 a top view of the fixing apparatus of FIG. 2 in an open state,

FIG. 5a, b, c various views of a first clamping jaw of the fixing apparatus,

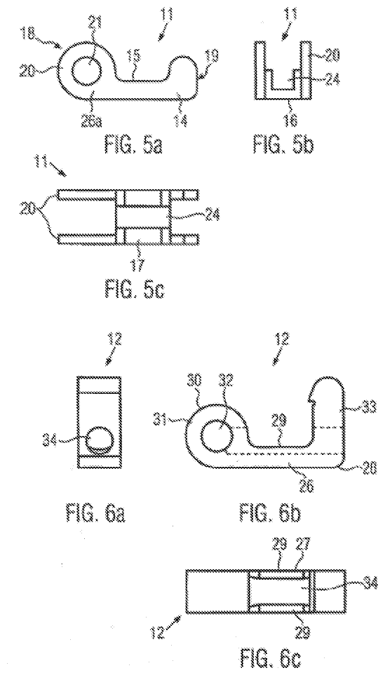

FIG. 6a, b, c various views of a second clamping jaw of the fixing apparatus,

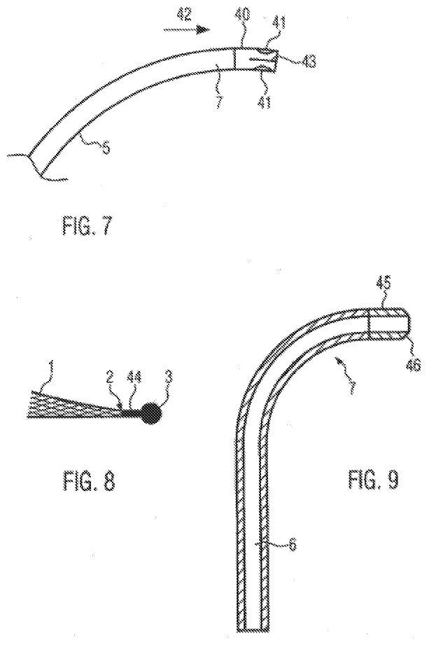

FIG. 7 a side view of the end portion of the insertion bar of FIG. 1 to which a bush is attached,

FIG. 8 a side view of the proximal end of the apnea stent of FIG. 1 with a spherical joining member,

FIG. 9 a sectional view of an end portion of the insertion tube of FIG. 1,

FIG. 9b a sectional view of an end portion of the insertion tube of FIG. 1 according to another embodiment thereof,

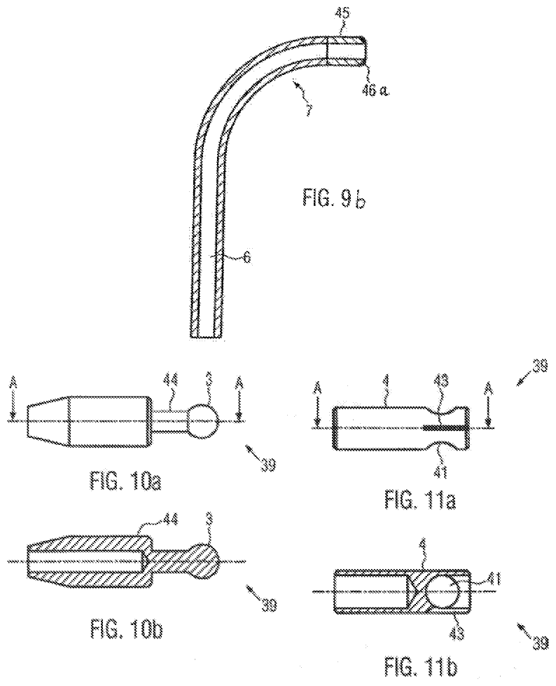

FIG. 10a a side view of a spherical joining member,

FIG. 10b a laterally cut view of the spherical joining member of FIG. 10a along line A-A,

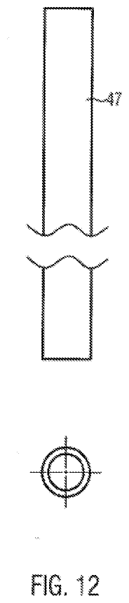

FIG. 11a another side view of the bush of FIG. 7, and

FIG. 11b another laterally cut view of the bush of FIG. 11a along line A-A, and

FIG. 12 a cleaning tube according to the invention.

An apnea stent 1 which is known from WO 2007/065408 A2 (FIG. 1c) to which reference is herewith made in its entirety is made in three sections.

A distal section of the stent forms an active part of the apnea stent 1. This distal section has a tubular shape and can be widened so as to keep open the respiratory duct.

A proximal section of the stent is provided for fixation of the stent in the nose region. The proximal section has the shape of a funnel and is widened from a proximal end to a distal end.

The distal and proximal sections are made as netting where wires and/or fibers or filaments are interlaced. It is only the term ‘wire’ that is used below, however, it shall also comprise fibers and filaments.

Each individual wire running towards the distal end of the stent is guided back to the proximal end of the stent at the distal end of the distal section. The thus produced bends are referred to as round ends. In the open state of the stent, they have a diameter ranging from about 1-2 mm. A distal end is thus provided at which no individual wire ends are exposed or have to be connected to one another by a further member. The wire or wires of the stent can be connected by crimping made at the proximal end of the stent. As a result of the round ends, injuries of the respiratory ducts by the distal end of the apnea stent are avoided.

A transitional section of the stent is provided for connecting the proximal section to the distal section. The transitional section is formed by twisting the wires of the stent into twisted strands. Each strand comprises at least two wires. The individual strands extend approximately in an axial direction of the apnea stent without interlacing. As a result, the transitional region has a high flexibility in the radial direction so as to effect a strong radial widening from the proximal section to the distal section within a short longitudinal extension. It is thus ensured that an approximately constant diameter forms over the entire length of the distal section.

A joining or coupling member 3 is arranged at the proximal end 2 of the apnea stent. Said member can be coupled to a joining or coupling member 4 of an insertion bar 5. The joining member 4 is arranged at one end of the insertion bar 5. In the present embodiment, the joining member 4 of the insertion bar 5 is formed as a bush and the joining member 3 of the apnea stent 1 is formed as a sphere which fits positively with the bush.

In order to introduce the apnea stent 1 into the respiratory duct, the apnea stent 1 is inserted in an insertion tube 6 by means of the insertion bar 5 and compressed. To this end, the insertion bar 5 is pushed through the insertion tube 6 so as to move the apnea stent 1 into the insertion tube 6 by the insertion bar 5.

Using this apparatus for treating snoring and/or sleep apnea, the stent 1 is inserted in one of the two nostrils in the state compressed in the insertion tube 6 and preferably pushed up to the epiglottis at the farthest where it is placed at its site of application. The stent 1 is released by retracting the insertion tube 6 and automatically expands into a predetermined size. As a result, the collapse of the pharynx and the occlusion of the respiratory ducts are prevented. Free air current and normal respiration are thus enabled. After the correct arrangement of the apnea stent 1 in the respiratory duct, the connection with the insertion bar is released.

This stent 1 is secured and/or fixed by means of a fixing apparatus 10 according to the invention to prevent an excessively deep penetration into, and slipping out of, the respiratory duct. The correct insertion depth of the apnea stent can be determined by means of this apparatus and the apnea stent 1 can be secured against unintended slipping into the respiratory duct.

The fixing apparatus 10 according to the invention comprises first and second clamping jaws 11, 12 which are connected to each other via a joint 13. The first and second clamping jaws 11, 12 form a fixing device (FIGS. 3, 4, 5a, b, c, 6a, b, c).

The first clamping jaw 11 is referred to as the base jaw below. The base jaw 11 has a rectangular base wall 14. The base wall 14 comprises a top side 15, a bottom side 16, and two side walls 17 extend upwards from the base wall 14 along the longitudinal sides thereof, and therefore the base wall 14 and the two side walls 17 define a U-shaped recess 24. The two opposite ends of the cuboidal base plate 14 are referred to as the joint side 18 and the locking side 19.

Two annular disk members 20 are formed at a distance from each other on the joint side 18 of the cuboidal base plate 14. They are flush with the outer surfaces of the side walls 17. The annular disk members 20 have a circular through opening 21 each; said openings are in alignment. The through openings 21 are designed to receive a tubular shaft 22 each. The tubular shaft is rotatably arranged in the through openings 21 and flush with the outer surfaces of the side walls. The first clamping jaw 11 is pivotably connected to the second clamping jaw 12 via the tubular shaft 22.

The side walls 17 extend at the locking side 19 beyond the front-end edge of the base wall 14 and therefore they define the U-shaped recess in a top view of the base jaw 11 (FIG. 4). The side walls are somewhat higher in this area.

The front-end edge of the base wall 14 on the bottom side 16 is referred to below as the locking edge 23.

A cuboidal fixing block 25 is arranged in groove 24. The cuboidal fixing block 25 protrudes approximately 3 mm from the top side 15 of the base wall 14. The cuboidal fixing block 25 is made of a plastic material, in particular silicone, and forms a fixing member.

The second clamping jaw 12 has an L-shape and a cuboidal cross-section. The second clamping jaw 12 comprises a long leg 26 and a short leg formed thereon at right angles, said leg being referred to below as the fixing leg 33. The long leg 26 has a base wall 26a including a top side 27 and a bottom side 28 and two side walls 29 which extend upwards from the top side 27 and define a groove between them.

The end of the long leg 26, which is opposite to the fixing leg 33, is referred to as the joint side 30. A tubular joint body 31 is formed on this joint side 30. The joint body 31 forms a tube having two side walls 29 and a circular through opening 32, the length of the tube corresponding to the inner width between the two annular disk members 20. The tubular shaft 22 is arranged in the through opening of the joint body. The tubular shaft 22 is rotatably supported in the through openings 21 of the annular disk members 20 and in the through opening 32 of the joint body 31.

The tubular shaft can also only be rotatably arranged in the through openings 21 of the annular disk members 20 or in the through opening 32 of the joint body 31. In each case, the tubular shaft is fixed so as to prevent it from automatically slipping out of the fixing apparatus.

A through hole 34 is formed in the fixing leg 33, said hole being in alignment with the groove of the long leg 26. A recess is made in the tubular joint body 31, said recess being also in alignment with the groove of the long leg 26. The through hole 34, the groove of the long leg 26 and the recess of the joint body 31 accommodate a fixing tube 35. The fixing tube 35 and/or the fixing member is made of plastic material, in particular silicone.

A latch 36 facing the joint body 31 is formed on the free end of the fixing leg 33. The latch 36 is made so as to grip behind the fixing edge 23 when the two legs are compressed. The latch 36 then locks behind the fixing edge 23 and holds the fixing apparatus 10 in a closed state. The latch 36 and the fixing edge thus form a locking member. In the locked state of the fixing apparatus, the base jaw 11 and the second clamping jaw 12 are arranged approximately parallel to each other, the fixing block 25 and the fixing tube 35 being pressed onto each other.

The proximal end 2 of the apnea stent 1 can be arranged between the fixing block 25 of the clamping jaw 11 and the fixing tube 35 of the clamping jaw 12. The proximal end 2 of the stent 1 is fixed in stationary fashion in the fixing apparatus 10 by pressing the tubular fixing tube 35 and the cuboidal fixing block 25 onto each other and deforming them. The proximal end of the apnea stent which is made of a nitinol netting is pressed flatly between the fixing tube 35 and the fixing block 25 and/or the fixing members and/or is deformed and fixed without being damaged or constantly deformed. Since the base jaw 11 and the clamping jaw 12 are clamped together in the locked state, the proximal end 2 of the apnea stent cannot get out of place.

The fixing block 25 and the fixing tube 35 form straight clamping edges or clamping faces without serration or the like. An apnea stent can thus be clamped between the base jaw 11 and the clamping jaw 1 several times without being damaged.

The locking member formed by the latch 36 and the fixing edge 23 is disposed outside the contact area between the two clamping members 11, 12. In the present embodiment, the locking member is arranged on the bottom side 16 of the base wall 14 of the base jaw 11 and thus on the side of the base jaw 11 which faces away from the contact area. As a result of the distance between the locking member and the contact area, there is only little risk that a portion of the apnea stent is clamped in the locking member between the latch 36 and the fixing edge 23, thus being damaged.

A contact pressure is defined by the dimensioning of the height of the fixing block 25 and the diameter of the fixing tube 35, which serves for fixing the proximal end of the apnea stent. While fixing, the fixing tube 35 is compressed by the fixing block. The smaller the height of the fixing block 25 and the smaller the diameter of the fixing tube 35, the lower the contact pressure. The greater the height of the fixing block 25 and the greater the diameter of the fixing tube 35, the higher is the contact pressure. In addition, the contact pressure can be adjusted by the hardness of the materials for the fixing block 25 and the fixing tube 35. The contact pressure results in a clamping force which has to be overcome to compress the base jaw and the clamping jaw into their locked state.

It is decisive for dimensioning that it is still easy for the user to compress the two clamping jaws, i.e. with acceptable expenditure of force. For this purpose, the user must overcome the clamping force and a locking force. The locking force is the force which is necessary to bend back the fixing leg such that the latch no longer grips behind the fixing edge. It thus depends on the resilience and the strength of the material from which the fixing leg is made. In order to open the fixing apparatus, it is only necessary to overcome the locking force.

Thus, the base jaw 11, the clamping jaw 12, the fixing block 25 and the fixing tube 35 have to be designed with respect to dimension, geometry and material so as to sufficiently fix the proximal end of the apnea stent without damage, and a user can also close the fixing apparatus simply and easily, above all with one hand. The contact pressure and the fixing force are variably adjustable via the dimensioning of the base jaw 11, the clamping jaw 12, the fixing block 25 and the fixing tube 35.

The geometry and the dimension of the two clamping jaws and the geometry of the latch also have an effect on the holding force. The dimensioning of the fixing apparatus follows from an optimum compromise between a sufficient size for the user-friendly handling of the fixing apparatus and from dimensions as compact as possible to impair the user as little as possible during sleep.

In particular, the clamping faces of the clamping jaws are formed so as not to damage the nitinol netting when the fixing apparatus is locked. The length of the clamping faces must correspond at least to the width of the compressed nitinol netting to avoid folding of the nitinol netting one on top of the other and thus damage. One or both surfaces of the clamping jaws are made as resilient faces. The surfaces are plane or planar or convex, i.e. they have no sharp edges so as not to damage the nitinol netting. It may also be provided for the surface of one of the two clamping jaws to have a rough surface including a structuring of up to 1/10 mm to secure the nitinol netting from getting out of place. It is possible to provide instead of the fixing block and the fixing tube made of silicone also closed-pore foams which are integrally connected to the clamping jaws. The contact pressure applied to the nitinol netting can be applied along a line or over part of or the entire surface of the clamping jaws. The apnea stent is arranged transversely to the clamping jaws in the fixing apparatus. The nitinol netting is thus clamped transversely to the longitudinal direction of the apnea stent.

During use, the fixing apparatus 10 abuts against the nose of a patient in the area of a nostril so as to safely and reliably prevent the apnea stent from an excessively deep slipping into the patient.

An attachment device 37 for the arrangement of the fixing apparatus at the patient's head can be disposed in the through opening or the support 32 to mount—the fixing apparatus and prevent the apnea stent from slipping out of the respiratory duct. The attachment device 37 can be made as a soft and/or elastic headband, for example, which extends through the cylindrical through opening or support 32 and is arranged around the head. It is also possible to use an attachment device which can be arranged like the temples of glasses or loops around a patient's ears. Since a through opening is provided as a support, the connection to the attachment device 37 cannot be opened or released by mistake. Another attachment device can be inserted in the form of a pin which is inserted in the through opening 32 in positive engagement and which has a projection protruding from the through opening, said projection being fixed to the user by means of a patch. This projection preferably has a plate-like shape.

In addition, a joining member 39 is provided which ensures a detachable connection between the insertion bar 5 and the proximal end 2 of the apnea stent 1. The joining member 39 comprises the joining member 3 on the proximal end 2 of the apnea stent 1 and the joining member 4 on the distal end of the insertion bar 5 (FIGS. 1a, 7, 8, 10a, 10b, 11a, 11b).

The insertion bar 5 is made of a plastic material (FIGS. 1a, 7). A tubular bush 4 is preferably formed in integral fashion on the distal end 7 of the insertion bar 5 (FIGS. 7, 11, 11b). The bush 4 has two oppositely arranged circular through openings 41. A slot 43 is provided in the longitudinal direction 42 of the insertion bar 5 in the region of the two circular through openings 41. The slot 43 extends to the distal end of the bush 4. The bush 4 can be formed directly on the distal end of the insertion bar 5 or can be made separately from another material and can be connected, e.g. adhered, in frictional engagement to the insertion bar 5.

At its proximal end 2, the apnea stent 1 has a sleeve 44 at the proximal end of which a spherical joining member 3 is formed (FIGS. 8, 10a, 10b). The spherical joining member 3 is made of stainless steel or a suitable plastic material, for example. Regarding the size, the diameter of the spherical joining member 3 corresponds almost to the outer periphery of the bush 4 formed on the insertion bar 5. The sleeve 44 is connected to the apnea stent 1 in a frictional engagement by means of adhesion, for example.

The spherical joining member 3 can be received between the two through bores 41 of the bush 4 of the insertion bar 5. A detachable connection is thus provided between the apnea stent 1 and the insertion bar 5. The longitudinal slot 43 formed in the bush 4 facilitates the insertion and the pulling of the spherical joining member 3 out of the bush 4. The joining or holding force of the joining member 39 can be adjusted via the ratio of the diameter of the spherical joining member 3 to the inner diameter of the bush 4 and via the hardness of the material for the bush 4 and the length of the slot 43. The spherical joining member 3 is supported in the bush 4 for free rotation. The advantage of such a design of the joining member 39 is that the apnea stent 1 orients itself in the insertion tube 6 easily and automatically into the optimum direction in accordance with its impressed shape or bend and the shape of the insertion tube. The shape of the insertion tube is adapted to the course of the nasal and pharyngeal zones. As a result, the insertion is facilitated and pain is avoided for the user since the shape of the insertion tube corresponds to the course of the nasal and pharyngeal zones.

The bush can also be formed on the apnea stent and the spherical joining member correspondingly on the insertion bar.

During the insertion, the insertion tube 6 is arranged above the apnea stent 1 to arrange the apnea stent in the respiratory duct. At its distal end, the insertion tube 6 preferably has a portion which is bent approximately in the shape of a segment, the proximal end of the insertion tube being substantially linear. Said portion extends over an angle of about 80° to 120° and has a radius of about 3 cm to 7 cm (FIGS. 1a, 9, 9b). The insertion tube is preferably made of a sufficiently hard, suitable plastic material, such as PEBAX having a shore hardness of 60 shore D to 80 shore D. The hardness of the plastic material has a considerable influence on an optimum and/or accurate adaptation of the insertion tube to the nasal passage during the insertion. An insertion tip 45 is integrally formed on the distal end of the insertion tube 6. The tip corresponds to the insertion tube 6 as regards both the inner diameter and the outer diameter and is formed flush therewith. The insertion tip 45 can be made of a flexible, substantially softer material, such as PEBAX having a shore hardness of 25 shore D to 45 shore D. The insertion tube 6 can also be made of one piece. Such a one-piece insertion tube can consist of a uniform material, such as PEBAX72, or it is possible to produce such a one-piece insertion tube by means of extrusion with a continuous material gradient. In the case of such an insertion tube there is a transitional region where the amount of one material constantly decreases and the amount of the other material correspondingly increases. The materials of such a tube are e.g. PEBAX72 in the region of the main body and PEBAX35 in the region of the insertion tip 45. As to the selection of the material, the above statements on the two-piece insertion tube are likewise applicable.

A chamfer 46 having an angle of approximately 45° is provided on the distal end of the insertion tip 45 of the insertion tube. Injuries of the vessel walls are thus avoided when the apnea stent 1 is inserted in the respiratory duct and the insertion is facilitated since the insertion tube can perfectly follow the bends of the respiratory duct and the insertion tip is very soft and flexible. The outer diameter of the insertion tube is up to 5 mm to enable an easy, pain-free insertion for the user. The inner diameter of the insertion tube is up to 4 mm to provide sufficient space for the reception of the apnea stent. An abutment against the pharyngeal zone is prevented by the curvature of the insertion tube so as to ensure an injury-free insertion.

Instead of the chamfer, the insertion tip 45 can also be made with a dome 46a. Such a dome can be made by means of thermal reshaping or by means of grinding or laser ablation, for example. Such a dome can be formed on the outer side and/or inner side.

The production of such a rounded tip is more complicated than a chamfer. However, the rounded tip provides more security when the stent is inserted. In addition, the resulting thin-walled end of a chamfer can harden since the plasticizers escape from the plastic material through the rather large surface area.

These shaped insertion tips 45 are atraumatic tips; the harder the material of the tip, the smoother and more round the shape should be.

The insertion of the apnea stent can additionally be supported when the rounded ends of the apnea stent 1 protrude somewhat (e.g. 2-4 mm) from the insertion tube 6 while inserting it. The rounded ends of the apnea stent 1 then form an elastic atraumatic insertion means serving for inserting the stent.

Having pulled the stent 1 out of the respiratory duct, it is contaminated with body fluids, such as nasal mucus. In order to clean the apnea stent 1, a cleaning tube 47 is provided. The cleaning tube 47 is preferably made of a plastic material. The diameter of the cleaning tube corresponds approximately to that of the portion of the apnea stent 1 having the greatest diameter in the widened state. The diameter of the cleaning tube 47 is preferably slightly smaller than that of the distal portion of the apnea stent 1 which has the greatest diameter when widened. The surface of the cleaning tube is preferably glazed. As a result of such a coating in the form of a glazing, the surface of the cleaning tube has a smaller surface adhesion and therefore the nitinol netting can be easily removed from the cleaning tube by pushing. However, the adhesion is sufficiently great to prevent automatic slipping of the apnea stent from the cleaning tube. The apnea stent 1 can be easily arranged above the cleaning tube 47 and cleaned under tap water. The cleaning tube 47 advantageously has a smooth surface so that no contaminations can adhere thereto. The cleaning tube can also be made as a rod of a solid material or as a pipe. Glass can also be provided as the material.

According to the invention, it is provided to clean the apnea stent 1 after the cleaning by means of a cleaning agent and/or a decontamination agent to prevent infections in the respiratory duct by carrying along germs adhering to the apnea stent 1.

A user may not get into contact with disinfecting chemicals, in particular in the nasal zone and pharyngeal zone. The apnea stent must be wetted completely, i.e. on both the inner face and outer face, with the decontaminant.

Therefore, the cleaning agent must effectively eliminate potential bacterial, fungal and viral contaminations but may not cause any irritant or toxic effects, in particular in the nasal and pharyngeal zones. Furthermore, it must be designed such that it wets as automatically and fully as possible the fine wires of the netting or gauze or mesh of the apnea stent 1. Therefore, classical disinfectants, such as Helipur®, Helix®, Stabimed®, etc. from B. Braun Melsungen or Gigasept Instru AF® from Schülke & Mayr GmbH which are usually based on aldehyde or phenol, are not suited. Enzymatic cleaning agents, such as Helizyme®, etc. from B. Braun Melsungen, have no decontaminating effect and therefore are not suited even though they are more skin-compatible. The current wound antiseptics PVP iodine and octenidine are not suited either because they have an irritating effect to the mucous membranes and a cell toxic effect.

A preparation which is known to be effective and well tolerated in man for wound treatment and infection prophylaxis (in particular also against multiresistant Staphylococcus aureus bacteria, “MRSA”) is polyhexanide solution having an optimized composition, e.g. Prontoderm®, Prontosan®, from B. Braun Melsungen or ProntoMan®, ProntoLind® from Prontomed GmbH.

Polyhexanide (-polyaminopropyl-biguanide) is a cationic biguanide having a broad microbiocidal effectiveness. It offers an effective reduction of germs and a broad spectrum of activity, even when used for a prolonged period of time. The microbiocidal effect is predominantly based on a selective influence of the permeability of the bacterial cell membrane by strong interaction with respect to acidic lipids of bacterial cell membranes. This leads to a good compatibility with the tissues in man.

The solution to be preferably used according to the invention comprises along with the polyhexanide active substance a betaine as the detergent in a suitable quantity. Such a polyhexanide solution which additionally contains undecaleneamidopropyl betaine has shown to be suitable for the decontamination of the apnea stent. The reduction of a bacterial test contamination is up to seven log levels and is thus above the known one of five log levels for the use in man. As a result of the betaine contained in the solution, the polyhexanide solution can be used very efficiently as a spray for the treatment of the apnea stent 1. The betaine reduces the surface tension to such a strong extent that the minimum amounts of sprayed-on polyhexanide solution cover the full surface of the wires, thus creating an effective decontamination also in particular on the inwardly facing side of the apnea stent 1, which is not directly sprayed. The dried test contamination having a large number of 10⁹ bacteria was reduced in cleaning tests by the polyhexanide solution by at least 5 log levels. Polyhexanide is preferably used in a concentration of 0.01% to up to 0.3%, more preferably of 0.1% to 0.3%, most preferably of 0.25%, to also achieve a disinfecting effect. The undecaleneamidopropyl betaine is preferably used in a concentration of 0.05% to 0.5%, more preferably 0.10% to 0.15%.

As a result of the cleaning method in three steps, including rinsing of the contaminants under warm water, mechanical cleaning on the cleaning tube and subsequent spraying with polyhexanide betaine solution, a disinfecting effect is achieved which enables the use of the apnea stent in various patients.

Alternative embodiments of the above described components are indicated below.

The clamping jaws 11, 12 of the fixing device of the fixing apparatus 10 according to the invention can also be made according to the principle of action of a Curry clamp. A Curry clamp comprises two rotatable plastic or metal jaws mounted on parallel axes, between which the proximal end of the apnea stent can be arranged. The jaws preferably have an eccentric shape and are pressed against each other by springs. In order to enhance the clamping action, their support surfaces have a structured profile.

When the apnea stent is arranged in the Curry clamp, it is secured against slipping into the respiratory duct since the jaws clamp the stent. For the purpose of release, the stent can be pulled upwards out of the clamp. However, the stent is then only secured against an excessively deep penetration into the respiratory duct.

The joining member 39 can also be made as a bayonet catch. The bayonet catch comprises two joining members, a first joining member being adapted to be arranged above a second joining member. The first joining member which is pushed over the second joining member has a longitudinal slot at the end of which a short transverse slot is disposed at right angles. The second joining member has a lug which is inserted in the transverse slot and then effects the firm connection. The connection is made via a plug-turn-movement. During joining the two joining members are placed one into the other. Elongate protrusions are mounted in both joining members at the joining point approximately perpendicularly to the plug-in direction. However, they do not run all around but are interrupted. Since the protrusions are now disposed with a slight inclination in the plane perpendicular to the plug-in direction, both joining members are pressed against each other by a rotary movement. Other sliding mechanisms are also possible.

A connection via a thread, a clip catch or a combination thereof is also possible.

The support can also be made as a clip catch or press button or the like.

What is claimed is:

1. A kit for treating sleep apnea and snoring, said kit comprising:
(a) an insertion tube effective for inserting an apparatus for treating sleep apnea and snoring through a nasal passage into a pharyngeal zone,
said insertion tube consisting of a substantially straight proximal portion, a circle segment distal portion, and an insertion tip integrally formed on the distal end of the circle segment distal portion of the insertion tube;
wherein the circle segment distal portion extends over an angle of about 80°-120° with a radius in a range of from about 3 cm to about 7 cm, the insertion tube being of length and inner and outer and diameter effective for inserting said apparatus for treating sleep apnea and snoring through said nasal passage into said pharyngeal zone, said inner diameter not exceeding 4 mm, and said outer diameter of not exceeding 5 mm;
wherein the substantially straight proximal portion and the circle segment distal portion are formed of a sufficiently hard plastic material to be effective for insertion of an apparatus for treating sleep apnea and snoring through said nasal passage into said pharyngeal zone, and
wherein the insertion tip is formed of a flexible soft material which is softer than the sufficiently hard plastic material of the substantially straight proximal portion and the circle segment distal portion; and
(b) an apparatus for treating sleep apnea and snoring, configured for use with the insertion tube so that said apparatus is insertable by the insertion tube through the nasal passage into the pharyngeal zone.

2. The kit of claim 1, wherein the apparatus for treating sleep apnea and snoring comprises an apnea stent.

3. The kit of claim 2, wherein said apnea stent has a distal section of tubular shape.

4. The kit of claim 2, wherein said apnea stent has a distal section comprising interlaced wires.

5. The kit of claim 4, wherein the interlaced wires at a distal end of the distal section of said apnea stent form round ends.

6. The kit of claim 5, wherein the apnea stent comprises a joining member at its proximal end.

7. The kit of claim 6, wherein the joining member is of spherical form.

8. The kit of claim 2, wherein the apnea stent is configured to be disposed in the insertion tube in a compressed state and to automatically expand to a predetermined size upon release from the insertion tube.

9. The kit of claim 2, further comprising a fixing apparatus configured for fixing the apnea stent in a respiratory duct.

10. The kit of claim 9, wherein the fixing apparatus comprises two clamping members between which a proximal end of the apnea stent can be fixed.

11. The kit of claim 2, further comprising one or more of an insertion bar, a joining member configured to connect the apnea stent and the insertion bar, and a cleaning tube configured to receive the apnea stent for cleaning.

12. The kit of claim 1, wherein the substantially straight proximal portion, the circle segment distal portion, and the insertion tip are formed as one piece.

13. The kit of claim 1, wherein the insertion tube comprises a transitional region where the amount of hard plastic material constantly decreases and the amount of flexible soft material correspondingly increases.

14. The kit of claim 1, wherein the circle segment distal portion of the insertion tube extends over an angle of about 90°-100°.

15. The kit of claim 1, wherein the hard plastic material of the insertion tube has a shore hardness of 60 shore D to 80 shore D.

16. The kit of claim 1, wherein the flexible soft material of the insertion tube has a shore hardness of 25 shore D to 45 shore D.

17. The kit of claim 1, wherein the hard plastic material of the insertion tube has a shore hardness of 60 shore D to 80 shore D, and wherein the flexible soft material of the insertion tube has a shore hardness of 25 shore D to 45 shore D.

18. The kit of claim 1, wherein the insertion tip of the insertion tube has a chamfer or a dome at its distal end.
List of Reference Signs

| | |
|---|---|
| 1 | apnea stent |
| 2 | proximal end |
| 3 | coupling member, sphere |
| 4 | coupling member, bush |
| 5 | insertion bar |
| 6 | insertion tube |
| 7 | distal end |
| 10 | fixing apparatus |
| 11 | first clamping jaw |
| 12 | second clamping jaw |
| 13 | joint |
| 14 | base wall |
| 15 | top side |
| 16 | bottom side |
| 17 | side walls |
| 18 | joint side |
| 19 | locking side |
| 20 | annular disk members |
| 21 | through opening |
| 22 | tubular shaft |
| 23 | locking edge |
| 24 | U-shaped recess |
| 25 | fixing block |
| 26 | long leg |
| 27 | top side |
| 28 | bottom side |
| 29 | side wall |
| 30 | joint side |
| 31 | joint body |
| 32 | through opening |
| 33 | fixing leg |
| 34 | through hole |
| 35 | fixing tube |
| 36 | latch |
| 37 | attachment device |
| 39 | joining member |
| 41 | through opening |
| 42 | longitudinal direction |
| 43 | slot |
| 44 | sleeve |
| 45 | insertion tip |
| 46 | chamfer |
| 46a | dome |
| 47 | cleaning tube |
